(19) United States
(12) Patent Application Publication
(10) Pub. No.: US 2026/0123844 A1
(43) Pub. Date: May 7, 2026

(54) Machine Learning-Based System and Method for Contactless Arterial Blood Pressure Waveform Monitoring

(71) Applicant: The Hong Kong University of Science and Technology, Hong Kong (CN)

(72) Inventors: Qingyong HU, Hong Kong (CN); Qian ZHANG, Hong Kong (CN); Yuxuan ZHOU, Shenzhen (CN)

(21) Appl. No.: 19/373,764

(22) Filed: Oct. 30, 2025

Related U.S. Application Data
(60) Provisional application No. 63/714,886, filed on Nov. 1, 2024.

(51) Int. Cl. A61B 5/021; A61B 5/00; G01S 13/02; G01S 13/89; G06N 3/0455; G06N 3/09

(52) U.S. Cl. CPC A61B 5/02108; A61B 5/7257; G01S 13/89; G06N 3/0455; G06N 3/09; G01S 2013/0245

(57) ABSTRACT

The present invention provides machine learning-based system and method for contactless ABPW monitoring. The method comprises: utilizing a mm Wave radar to transmit a mmWave signal waveform and to receive reflection signals from a subject's chest; extracting complex-valued mmWave IQ data from the received reflection signals; utilizing a beamforming-based data augmentation module to steer the complex-valued mmWave IQ data to form target signal beams; and utilizing a mmWave-ABPW transformer to estimate ABPW from the target signal beams. During training stage, the beamforming-based data augmentation module is configured to generate mm Wave signal beam data from different angles for training the mmWave-ABPW transformer; and a cross-modality knowledge transfer module is utilized to generate a teacher model to co-supervise training of the mmWave-ABPW transformer.

### CROSS-REFERENCE TO RELATED APPLICATIONS

The present application claims priority from the U.S. Provisional Patent Application No. 63/714,886 filed Nov. 1, 2024, and the disclosure of which is incorporated herein by reference in its entirety.

### FIELD OF THE INVENTION

The present invention generally relates to machine learning technology, and more specifically relates to machine-learning-based system and method for contactless arterial blood pressure waveform monitoring.

### BACKGROUND OF THE INVENTION

Blood pressure (BP) monitoring is vital to assess the health status of the heart and cerebral vessels. While discrete systolic/diastolic blood pressure (SBP/DBP) values are commonly measured, they are insufficient for detailed assessment of cardiac indicators (e.g., stroke volume, cardiac output (CO), and vascular resistance), which are associated with prevalent cardiac diseases like heart failure and cardiogenic shock that affect more than 64 million people around the world.

Beyond discrete values, the arterial blood pressure waveform (ABPW) contains finer-grained information that depicts the complete cardiac cycle, including the rise of blood pressure due to the blood ejection in the systole stage, the descent at the closure of the aortic valve, and the trough state when blood flows out of the aorta as shown in FIG. 1. With detailed BP variations inside heartbeats, ABPW can also depict abnormalities with internal cardiovascular statuses beyond discrete BP values, some of which may not even be reflected in ECG signals, and offer comprehensive insights into cardiovascular health compared to discrete blood pressure measurements. FIG. 2 shows several cases of cardiac abnormalities that may have similar discrete BP values but behave quite distinctly in ABPW. Continuous ABPW monitoring is significant for assessing the overall cardiovascular status to help diagnose relevant cardiac diseases.

Existing ABPW monitoring methods require invasive procedures or continuous skin contact, which are inconvenient and uncomfortable. The arterial catheter-based method inserts a tube into blood vessels. It is a clinical gold standard method but is limited to intensive care unit (ICU) scenarios due to its invasiveness. For non-invasive methods, some prior works, including cuff-based auscultatory and oscillometric methods, wearable methods, contactless solutions, etc., estimate discrete SBP/DBP values as coarse approximations of ABPW, missing essential information on variation trends. Nonetheless, existing contactless solutions all fail to achieve continuous ABPW monitoring, because they require a period of clean signals (several cardiac cycles) to summarize one discrete result based on the pulse transit time (PTT) methodology.

Recently, some progress in ABPW monitoring, including the volume clamp method and wearable methods has been made to obtain ABPW in a non-invasive manner. However, they require either dedicated and expensive hardware (˜40K USD) or close skin contact, leading to discomfort and inconvenience in daily scenarios.

On the other hand, ABPW is found relating blood volume changes in the vessels and electrical cardiac activities. FIG. 3 exemplifies ABPW during a single cardiac cycle. In the systole stage, the left ventricle ejects blood into the aorta. The kinetic energy of the ejected blood forces the elastic aortic wall to expand, causing an increase in blood pressure. When the aortic valve closes, the previously ejected blood returns to the heart, resulting in a reduction of blood pressure. The highest, lowest, and mean values of the waveform correspond to SBP, DBP, and MAP, respectively. ABPW encompasses vital cardiac information that holds clinical significance. For example, the slope of the ascending waveform reflects myocardial contractility while the slope of the downstroke waveform is related to systemic vascular resistance that is associated with the stress developed in the left ventricular during ejection. The systolic area and diastolic area reflect the ventricular wall stress and contractility. The cumulative area under the ABPW curve (AUC) exhibits a strong correlation with CO, a pivotal measure for evaluating circulatory performance and preventing heart failure. FIG. 2 illustrates several abnormal cases of ABPW. For example, aortic regurgitation is characterized by its bifid waveform is caused by abnormal blood circulation which blood pumped out of the left ventricle leaks backward.

Some methods have been using mm Wave signals to reflect chest vibrations caused by cardiac activities. However, it is not straightforward to obtain ABPW from mmWave signals. As ABPW requires accurate estimation for both pressure values and waveform shapes while ECG/PPG/SCG-like signals only require accurate shapes, it is more challenging for contactless ABPW monitoring. From the technique aspect, previous works are mainly based on neural networks with one-level bottleneck with a coarse resolution.

Due to the complexity of the cardiac kinetic system and the temporal inherence inside ABPW, it is hard to derive sequence-to-sequence (seq-to-seq) expressions from mmWave to ABPW to preserve the waveform details. Besides, the nature of physiological diversity across individuals makes it harder to fit into every subject.

Since ABPW relies on temporal-context analysis with a high sampling rate requirement (e.g., 125 Hz in our setting), this seq-to-seq mapping requires informative sequential mmWave inputs. Moreover, due to the small wavelength, mmWave signals are highly sensitive to disturbances. Even millimeter-level motions may introduce large and non-linear distortions, which makes it challenging to acquire reliable sequential mmWave data. Some existing contactless discrete BP estimation approaches perform data cleaning across multiple cardiac cycles to ensure performance, and thus cannot satisfy the ABPW sampling rate requirement.

### SUMMARY OF THE INVENTION

In accordance with the first aspect of the present invention, a machine learning-based system for contactless ABPW monitoring is provided. The system comprises: a mm Wave radar; one or more processors in communication with the mm Wave radar; machine learning tools in communication with the one or more processors; a memory device storing the machine learning tools and inter-operably coupled with the one or more processors; wherein: the machine learning tools include an mmWave-ABPW transformer and a beamforming-based data augmentation module; the mmWave radar is configured to transmit a mmWave signal waveform and to receive reflection signals from a subject's chest; the memory device further stores instructions that, when executed, cause the one or more processors to: extract complex-valued mmWave IQ data from the received reflection signals; utilize the beamforming-based data augmentation module to steer the complex-valued mmWave IQ data to form target signal beams; and utilize the mmWave-ABPW transformer to estimate ABPW from the target signal beams.

In accordance with a second aspect of the present invention, a machine learning-based method for contactless ABPW monitoring is provided. The method comprises: utilizing a mm Wave radar to transmit a mmWave signal waveform and to receive reflection signals from a subject's chest; extracting complex-valued mmWave IQ data from the received reflection signals; utilizing a beamforming-based data augmentation module to steer the complex-valued mm Wave IQ data to form target signal beams; and utilizing a mmWave-ABPW transformer to estimate ABPW from the target signal beams

Preferably, the transmitted mmWave signal waveform is a frequency modulated continuous waveform; and the mmWave radar is further configured to mix the received reflection signals with the transmitted mmWave signal waveform to obtain intermediate frequency signals.

Preferably, the one or more processors are configured to: apply range-fast Fourier transformation on the intermediate frequency signals to obtain frequency and phase of the intermediate frequency signals; detect the signal regions of the subject's chest from the frequency and phase of the intermediate frequency signals using a constant false alarm rate algorithm; search range bins with largest reflection energy from the detected signal regions; and extract the complex-valued mm Wave IQ data from the searched range bins.

Preferably, the mmWave-ABPW transformer includes a ABPW feature extractor comprising: an encoder configured to encode the target signal beams and generate a compressed feature tensor; a self-attention transformer configured to perform global context modelling and spatial attention refinement on the compressed feature tensor to obtain a refined feature tensor; a decoder configured to decode the refined feature tensor and generate an ABPW feature tensor; and a plurality of spatially-aware attention modules bridging intermediate layers of the encoder and the decoder to facilitate the ABPW feature extractor to concatenate feature tensors at different spatial dimensions by selectively re-weight feature activations according to importances of the target signal beams from different reflection areas.

Preferably, the mmWave-ABPW transformer further includes a personalization waveform regressor comprising: a plurality of expert regressor models trained respectively with a plurality of training data sets corresponding to respective blood pressure categories; and an explicit gate configured to select an appropriate expert regressor model for ABPW estimation based on a coarse blood pressure category of the subject.

Preferably, the appropriate expert regressor model is further trained using ABPW data of the subject.

Preferably, the beamforming-based data augmentation module comprises: a first neural network configured to estimate heart rate of the subject from the complex-valued mm Wave IQ data; and a second neural network configured to identify spectral features of the complex-valued mmWave IQ data and identify the target signal beams based on the estimated heart rate and the identified spectral features.

Preferably, the beamforming-based data augmentation module is configured to generate mm Wave signal beam data from different angles for training the mmWave-ABPW transformer.

Preferably, the machine learning tools further include a cross-modality knowledge transfer module configured to generate a teacher model to co-supervise training of the mmWave-ABPW transformer.

Preferably, the teacher model is a transformer model trained on a public ECG/PPG dataset to predict ECG/PPG signals from mm Wave data.

The present invention demonstrates remarkable performance in both detailed shapes and values under challenging scenarios. The evaluation results prove that our system achieves accurate and robust ABPW estimation and holds great potential for cardiovascular status monitoring, cardiac abnormality detection and long-time monitoring, as evidenced by our case studies.

Compared to existing contactless cardiac sensing technologies, the present invention extends the scope of contactless cardiac sensing as arterial blood pressure waveform can describe detailed cardio-dynamics as it can directly indicate abnormal blood pressure like hypertension and abnormal cardiovascular conditions. The present invention adopts a hybrid transformer featured with multiple resolution aggregation that could benefit more comprehensive temporal analysis. Besides, the present invention reduces the requirement of high-quality data with the theoretically guaranteed beamforming-based data augmentation and compensates for extra information from other modalities with cross-modality knowledge transfer.

### BRIEF DESCRIPTION OF THE DRAWINGS

Embodiments of the invention are described in more details hereinafter with reference to the drawings, in which:

FIG. 1 shows how arterial blood pressure waveform (ABPW) depicts cardiac activities and fine-grained cardio-dynamic indicators, e.g., cardiac output. SBP and DBP are systolic blood pressure and diastolic blood pressure as the maximum and minimum values of the waveform, respectively.

FIG. 2 illustrate cardiac abnormalities that can be reflected in ABPW but behave similarly in discrete SBP/DBP.

FIG. 3 shows an example of ABPW.

FIGS. 4A and 4B illustrate respectively schematic diagrams various stages of a method for contactless monitoring of ABPW using mmWave signals in accordance with one embodiment of the present invention respectively.

FIG. 5 shows a block diagram of a contact less ABPW monitoring system for implementing the method of FIGS. 4A and 4B.

FIGS. 6A to 6D show transmission and reflection signals, intermediate frequency signals, range-FFT operation, and complex-valued IQ signal extraction during the data collection process, respectively.

FIG. 7 illustrate correlation among cardiac signals (ECG/PPG/SCG), mm Wave signals, and ABPW, where MC refers to the closure of the mitral valve, i.e., the beginning of the systole phase, and AC denotes the closure of the aortic valve, i.e., the end of the systole phase and the beginning of the diastole phase.

FIG. 8A shows architecture of a feature extractor of a deep learning mmWave-ABPW transformer (mmFormer) in accordance with one embodiment of the present invention; FIG. 8B shows an example of extracted features.

FIG. 9 shows architecture of a personalization waveform regressor of the mmFormer.

FIGS. 10A and 10B illustrate I/Q data of signals augmented with different beamforming angles. FIG. 10C shows the I/Q data for non-beamforming signal. FIGS. 10D and 10E illustrate phase waveforms of signals augmented with different beamforming angles. FIG. 10F shows phase waveform for non-beamforming signal.

FIG. 11 shows an algorithm for beamforming-based data augmentation accordance with one embodiment of the present invention.

FIG. 12 illustrates schematic diagram of the training scheme for the mmFormer based on a cross-modality knowledge transfer (CMKT) framework in accordance with one embodiment of the present invention.

FIG. 13 shows relative cardiac output means obtained in a case study of calculating relative Cardiac Output through ABPW (Case study 1).

FIG. 14A shows a ABPW of a reference subject with chronic aortic regurgitation (AR) from a medical reference, FIG. 14B shows estimation of WaveBP for a subject in hypertension stage-1 with AR in a case study of cardiac abnormality detection through ABPW (Case study 2). y is the ABPW ground truth and ŷ is WaveBP's estimation (Type II). ECG is the corresponding electrocardiogram signal, which may not be non-specific to AR.

FIG. 15 shows ABPW estimation in a case study of long-time ABPW monitoring (Case study 3). SBPₚ, DBPₚ, and SBP_{g}, DBPₚ are discrete SBP/DBP extracted from the ABPW estimations and the corresponding ground truth, respectively. ŷ and y are the WaveBP's ABPW estimation and ground truth at the 7th minute, respectively.

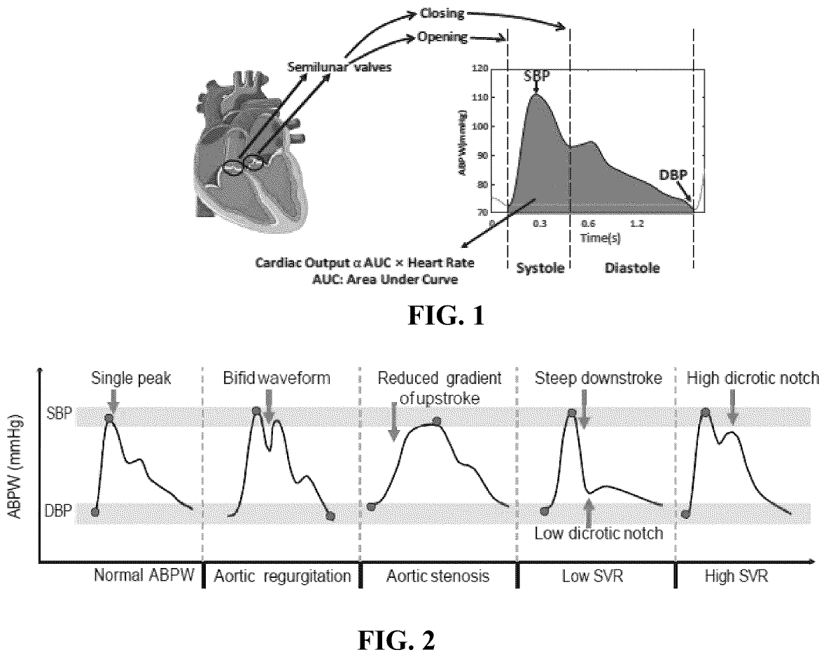

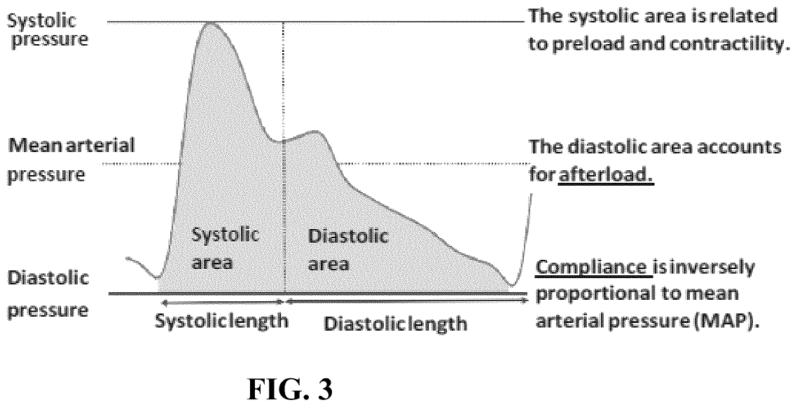

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

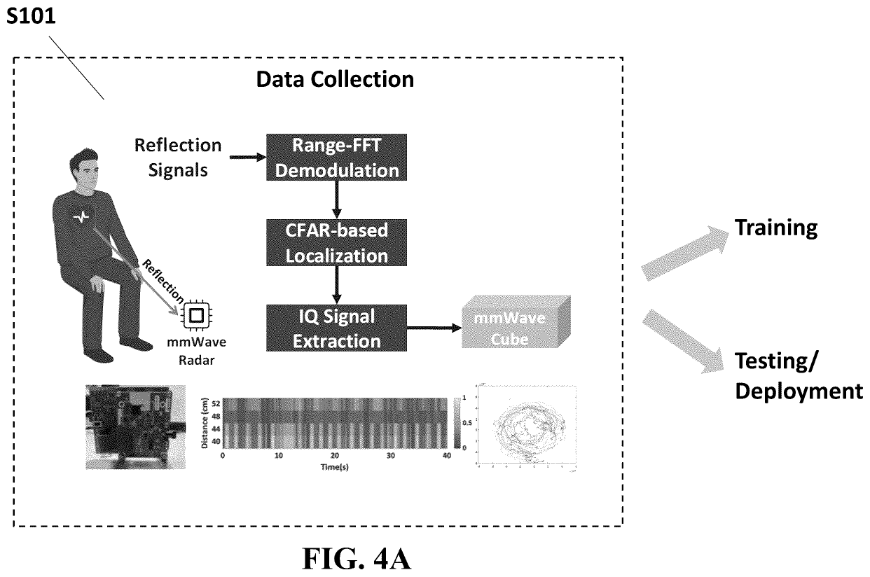

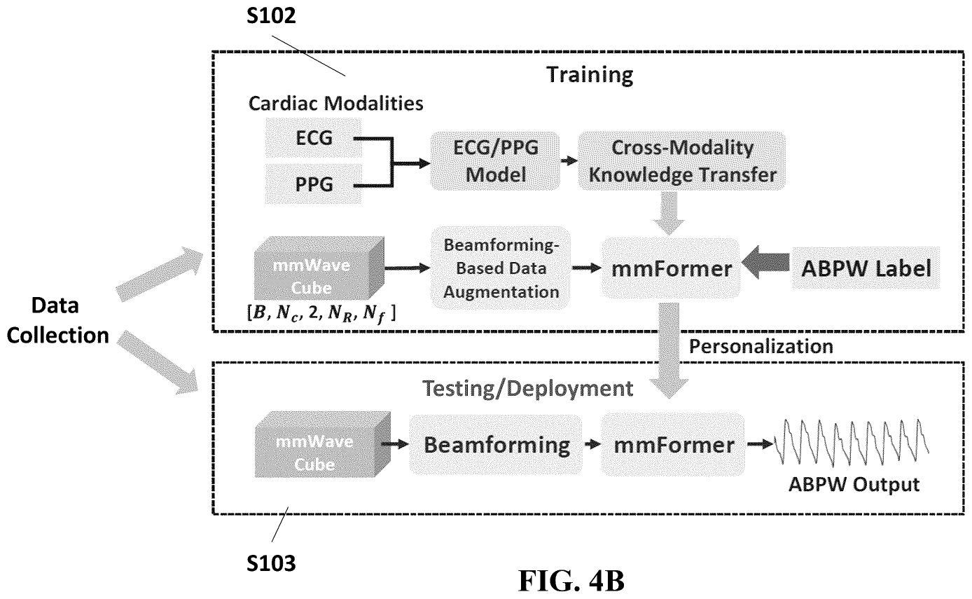

FIGS. 4A and 4B are schematic diagrams of different stages of a method S100 for contactless monitoring of ABPW using mmWave signals in accordance with one embodiment of the present invention respectively. As shown, the method S100 comprises three stages: S101, real-world mmWave data collection and data cube extraction; S102, training of machine learning tools; and S103, deployment of the machine learning tools to estimate ABPW from the mmWave data.

In stage S101, a mmWave radar is utilized to transmit a mmWave signal waveform and to collect multi-channel reflection signals from a subject's chest; and mmWave data cube carrying cardiac activity information of a subject are extracted, as will be described in more details later, from the collected reflection signals.

In stage S102, a machine learning tool mmFormer is trained, as will be described in more details later, to estimate ABPW from mmWave data by utilizing a Beamforming-based Data Augmentation (BeamDA) module and a Cross Modality Knowledge Transfer (CMKT) module.

In stage S103, mmWave data is fed to a beamforming module to steer the complex-valued mm Wave IQ data to form target signal beams and the target signal beams are fed to the mmFormer to extract ABPW.

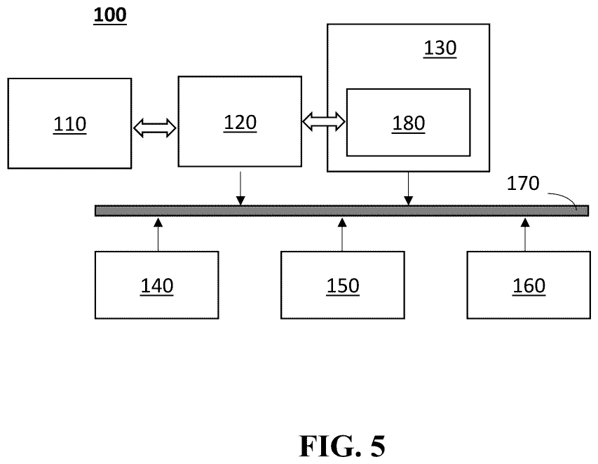

FIG. 5 illustrates a block diagram of a system 100 for contactless ABPW monitoring. The system 100 comprises a mm Wave radar 110, one or more processors 120, a memory device 130, an input interface 140, an output interface 150, and a communication module 160. As shown, these components are operatively connected through a bus 170.

The mmWave radar 110 may be a commercial off-the-shelf millimeter-wave radar including an antenna array configured to transmit mmWave signals to a chest of the subject and receive mmWave reflection signals from the chest of the subject. The antenna may be a phased antenna array, consisting of antenna elements spaced at sub-wavelength distances. Each element captures a slightly different version of the incoming wave due to path length difference.

The one or more processors 120 may be electrically coupled with the mmWave radar and configured to preprocess the mm Wave reflection signals to obtain the mm Wave data cubes containing hemodynamic related information correlating the cardiac activity information and ABPW.

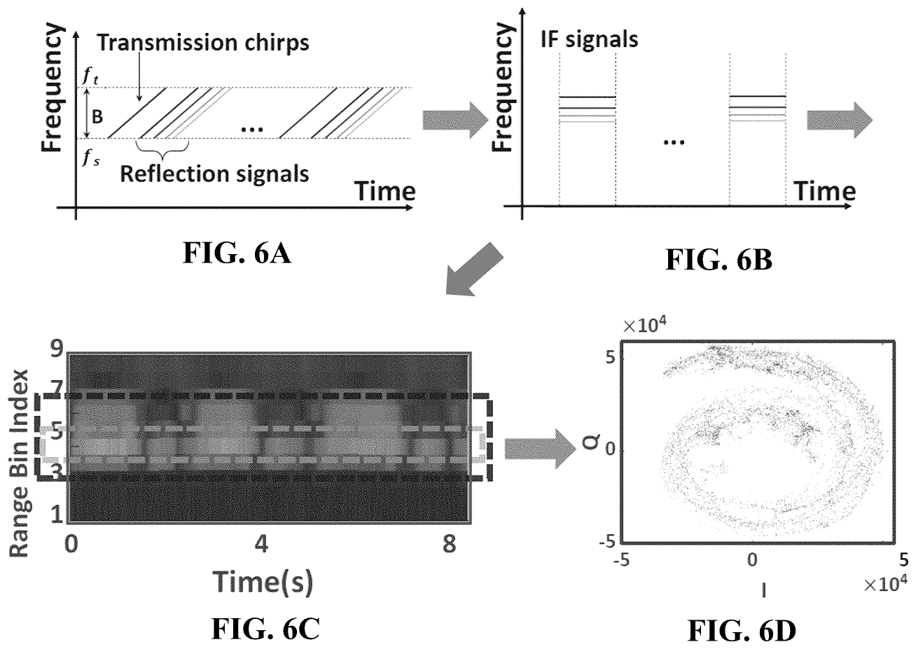

More specifically, during the data collection stage S101, the mmWave radar 110 is configured to transmit frequency-modulated continuous waveform (FMCW) as shown in FIG. 6A, i.e., chirps, on to the subject's chest, and collects reflection signals from the subject's chest via the antenna array. The received signals are mixed with transmitted signals to obtain intermediate frequency (IF) signals as shown in FIG. 6B. The intermediate frequency (IF) signals X_{IF} may be expressed as:

$${X}_{IF} ( i , t ) \approx {α}_{i} exp [ - j 4 π ( {f}_{c} + Bt / {T}_{c} ) d ( t ) / c ] , \qquad ( 1 )$$

where i denotes i-th reflection path channel. c is the light speed. αᵢ is the complex path loss for channel i. f_{c} denotes the starting frequency of chirp signals. B denotes the bandwidth of sweeping frequency. T_{c} denotes chirp duration. d(t) denotes the distance from the sensed object, e.g., the human chest to the mmWave radar.

By applying range-FFT operation on the IF signals X_{IF}, the time-domain sampled complex IF signal data are converted to frequency-domain. Each chirp/frame is processed to obtain a range FFT spectrum. The frequency and phase of IF signals can be obtained by:

$$f = 2 Bd ( t ) / ( {cT}_{c} ) , ϕ ( t ) = 4 π d ( t ) / ( c / {f}_{c} ) . \qquad ( 2 )$$

Based on the above equations, the IF signal, with a bandwidth of 4 GHZ, enables object localization with a resolution of

$$\frac{c}{2 B} \approx 4 cm .$$

Moreover, it can detect micrometer-level distance changes through the phase φ(t) and could be utilized to sense fine-grained cardiac activities. The complex-valued representations of mmWave range-FFT results are used to preserve information from both amplitude and phase aspects.

Furthermore, a Constant False Alarm Rate (CFAR) detection algorithm is used to locate the potential region of the human chest and then search the range bin with the largest reflection energy as shown in the dashed area in FIG. 6C. Multiple adjacent reflection bins are extracted as the input data X∈ [symbol] Here, N_{c}, N_{b}, and N_{f} are the number of wireless channels between the transmitting and receiving antennas, the number of selected bins in range-FFT results and the number of frames, respectively, and the number of 2 represents the real and imaginary parts of the complex signals. FIG. 6D is an example of complex-valued mmWave IQ data extracted from one range bin.

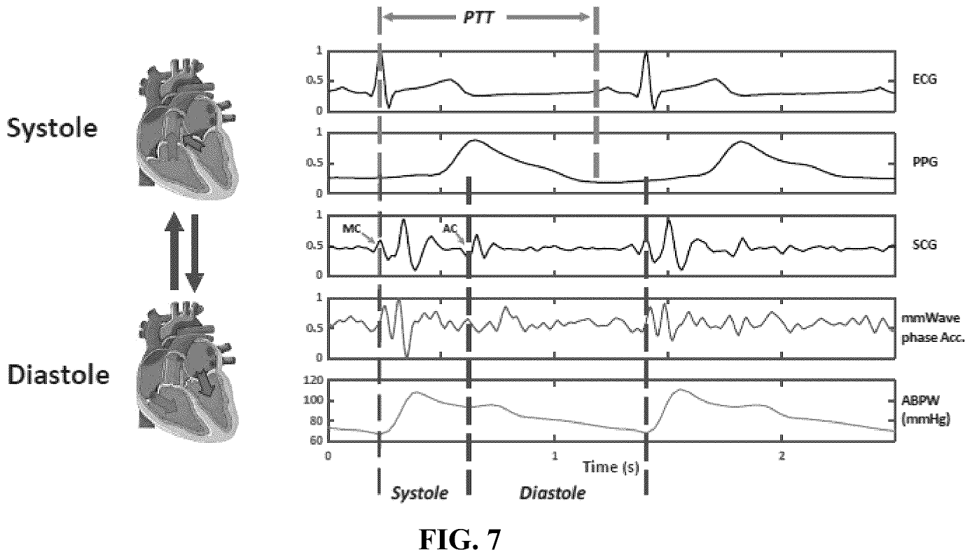

The mmWave reflection signals can capture fine-grained chest movements induced by heart pulsations during the systole and diastole phases. Compared with the heart pulsations captured in seismocardiogram (SCG) with an IMU sensor on the chest, FIG. 7 shows that the accelerations of the mm Wave phases exhibit remarkable similarity with SCG signals, thereby confirming the presence of detailed cardiac information within mmWave signals.

Simultaneously, the pulsations of the heart during the systole and diastole phases also lead to variations in blood flow I (t) within the blood vessels, consequently causing fluctuations in blood pressure P (t), i.e., ABPW. The quantitative relationship between I (t) and P (t) can be described by the widely utilized three-element Windkessel model in hemodynamics modeling:

$$( I + \frac{{R}_{1}}{{R}_{2}} ) I ( t ) + {CR}_{1} \frac{dl ( t )}{dt} = \frac{P ( t )}{{R}_{2}} + C \frac{dP ( t )}{dt} \qquad ( 3 )$$

The two terms on the left-hand side represent the incoming blood volumes pumped from the heart and flowing out of the artery. The first term on the right-hand side represents the blood flow into the heart, while the second term represents the stored volume changes inside the artery, which can be observed through PPG signals. Besides, R₁, R₂ and C represent individual-specific parameters, specifically denoting the aortic characteristic impedance, peripheral resistance on blood flow, and arterial compliance, respectively.

Furthermore, based on the Moens-Korteweg equation and Hughes Equation, the initial state of P(t), denoted as P(0) or DBP, has a significant correlation with the pulse transit time (PTT) as demonstrated in the following equation:

$$P ( 0 ) = \frac{1}{η} [ 2 ln ( \frac{L}{PTT} ) + ln ( \frac{2 ρ r}{{Eh}_{0}} ) ] \qquad ( 4 )$$

PTT can be approximately acquired from ECG and PPG signals depicted in FIG. 7. L denotes the distance of blood propagation from two measured sites. η, h, r, ρ, and E₀ are individual parameters such as the artery material coefficient, the artery thickness, the artery radius, and the elasticity modulus of the vessel wall at zero pressure, respectively.

Equation 3 and 4 illustrate the pressure value correlation while FIG. 7 demonstrates the temporal correlation between ABPW and cardiac signals.

The memory device 130 is inter-operably coupled with the processors 120 and configured to store and retrieve information. The memory device 130 may include volatile memory such as random-access memory, non-volatile memory such as read-only memory, and persistent storage devices. Examples of persistent storage include flash memory, solid-state drives, or similar storage media.

The input interface 140 enables reception of input signals by the system 100. Such signals may be generated by user interactions. The input interface 140 may connect the system 100 with one or more input devices, such as a touchscreen, a keyboard, or a trackball. In some configurations, part or all of the input interface 140 may be built directly into an input device. For example, the interface may be embedded within a touchscreen unit.

The output interface 150 provides output signals from the system 100. These output signals may correspond to information presented to a user. The output interface 150 links the system 100 with various output devices, such as a display (e.g., an LCD or touchscreen display), a speaker, indicator lights (such as LEDs), or a printer. In some cases, the output interface 150 may be incorporated directly within an output device, for instance, a display module.

The communication module 160 facilitates interaction between the system 100 and external devices or communication networks. This module enables sending and receiving of communication signals, which may follow particular standards or protocols. For example, the communication module 160 may provide connectivity via cellular data networks such as GSM, CDMA, EVDO, LTE, or similar technologies. Additionally, or alternatively, it may enable communication using Wi-Fi™, Bluetooth™, near-field communication (NFC), or a combination of these. NFC may also support functions such as contactless payments. In certain embodiments, the communication module 160 may be integrated into another component of the system 100, such as a dedicated communications chipset.

Executable instructions are run by the processors 120 from a computer-readable medium. For example, instructions may be transferred into random-access memory from persistent storage within the memory device 130, or executed directly from read-only memory.

The memory device 130 is configured to store at least one operating system and one or more application programs. The operating system provides access for the application programs to the hardware resources of the system 100, including the processors 120, the memory device 130, the input interface 140, the output interface 150, and the communication module 160. Suitable operating systems may include Apple™ OS X, Android™, Microsoft™ Windows™, Linux distributions, or equivalents.

The system 100 further include machine learning tools 180 in communication with the one or more processors. The pre-trained machine learning tools may be stored in and retrievable from the memory device 130. The memory device 130 is inter-operably coupled with the processors 120 and storing instructions that, when executed, cause the one or more processors to utilize the machine learning tools to perform operations for contactless ABPW monitoring.

mmFormer

The mutual cardiac information shared between the heart pulsations captured in mmWave reflections and ABPW serves as the primary motivation to develop a contactless mmWave-based system for estimating ABPW. ABPW requires sequential mapping and temporal-context analysis. To this end, the machine learning tools 180 include the deep learning mmWave-ABPW transformer (denoted as mmFormer) pretrained to extract ABPW from mm Wave signals carrying cardiac activity information.

The mmFormer may comprise a feature extractor configured to process an input tensor derived from a millimeter-wave (mmWave) signal and generate a structured feature representation suitable for downstream analysis and a personalized waveform regressor such that the correlations between mm Wave signals and ABPW can be effectively captured.

The feature extractor is a hybrid transformer with spatially-informed shortcuts at multiple temporal resolutions which implicitly learns ABPW sequences from spatial reflections from the subject's chest with multi-resolution awareness. It enables consistent sequence-to-sequence transformations while accommodating different levels of personalization efforts. Since ABPW requires a detailed temporal mapping with the whole cardiac cycle analysis, to facilitate sequential mapping from mmWave to ABPW, instead of aggregating multiple sequential mmWave reflections, prior knowledge is imposed as a temporal consistency regularization that this sequential translation intends to be consistent across different temporal resolutions.

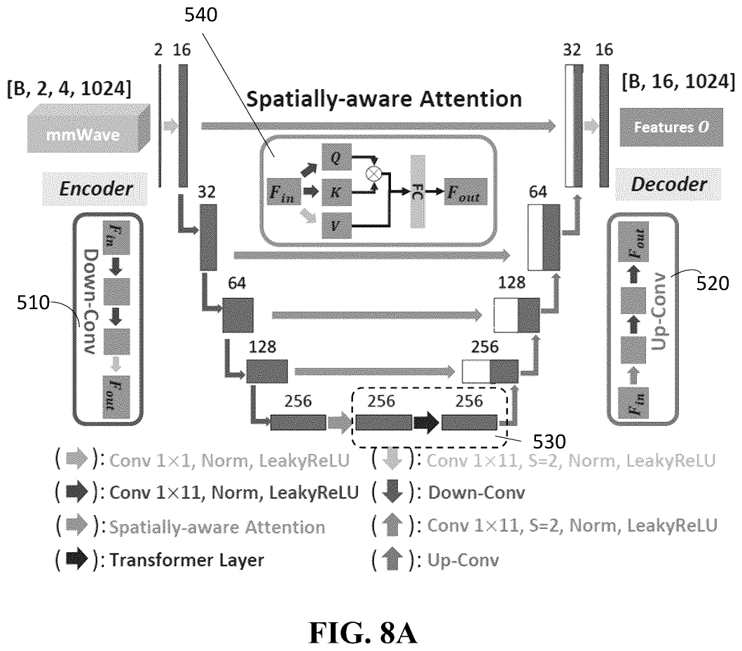

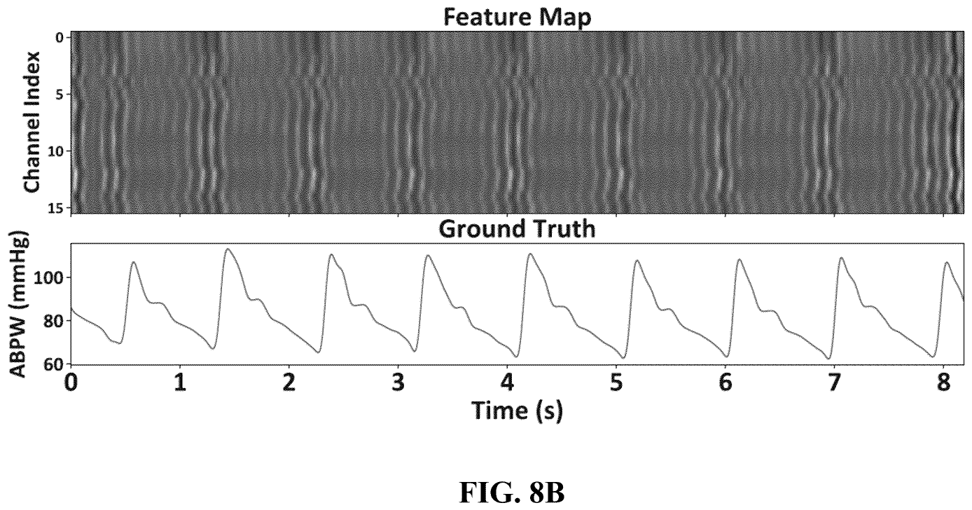

FIG. 8A shows an example architecture of a feature extractor 500 in accordance with one embodiment of the present invention. As shown, the feature extractor 500 has a hybrid UNet-transformer architecture featured with spatially-informed multi-resolution awareness shortcuts for effective temporal mapping.

The feature extractor 500 comprises an encoder 510, a decoder 520, a self-attention transformer 530 connected between a final layer of the encoder 510 and a first layer of the decoder 520; and a plurality of spatially-aware attention modules 540 bridging intermediate layers of the encoder 510 and the decoder 520.

The encoder 510 may comprise a sequence of down-convolution modules configured to progressively compress the spatial/temporal resolution of the input tensor while expanding its feature dimension. Each down-convolution module includes a convolution layer (Conv 1×11 or Conv 1×1), a normalization layer, and a LeakyReLU activation function. Certain down-convolution modules are further configured with stride=2, thereby reducing resolution while increasing channel depth.

For example, when an input mmWave signal tensor of shape [B, 2, 4, 1024] is received, where B is the batch size, 2 represent number of channels, 4 may represent antenna elements or spatial bins, 1024 is the temporal or frequency resolution, the encoder 510 is configured to encode the mmWave signals using convolution operations with 5 blocks corresponding to different time resolutions. Each layer encodes the mmWave signals and downsamples them for the subsequent layer with the convolution kernels with a stride of S=2, and generate intermediate feature maps with progressively larger feature dimensions and outputs a compressed feature tensor having 256 channels.

The self-attention transformer 530 is configured to perform global context modelling and spatial attention refinement. The transformer 530 may comprise a transformer layer configured to receive the 256-channel feature tensor and compute long-range dependencies across the spatial and temporal dimensions.

The decoder 520 is configured to upsample the downsampled embeddings from the lower layers using transposed convolution kernels and combine them with the spatially-informed feature representations passed from the encoder through the spatially-aware attention module at the same layer.

The spatially-aware attention modules 540 act as spatially-informed shortcuts bridging intermediate layers of the encoder 510 and the decoder 520. Different from typical UNet models that both the input and the output share the same dimension, the spatially-informed shortcuts enable the mmFormer to dynamically aggregate multiple mmWave reflections from the human chest to derive the target ABPW features.

Each spatially-aware attention module 540 is incorporated into each layer of mmFormer to facilitate information delivery from the encoder's perspective to the decoder and configured selectively re-weight feature activations according to spatial importance to enhance spatially-aware representation through a query-key-value mechanism.

More specifically, given the features extracted from multiple reflection signals as [symbol] , where D represents the dimension of feature channels, N_{b} represents the number of reflected areas, and T represents the time length of the sequence, the aggregated result Fₒᵤₜ can be calculated:

$$Q = f ( {ConV}_{Q} ( {F}_{in} ) ) , {K}_{T} = f ( {ConV}_{K} ( {F}_{in )} ) , V = f ( {ConV}_{V} ( {F}_{in} ) ) \qquad ( 5 )$$

$$C = Softmax ( \frac{QK}{\sqrt{D}} ) , {F}_{out} = FC ( CV )$$

where Q, K, and V represent the calculated query, key, and value in the attention module, respectively. Convᵢ denotes the convolution operator for i∈{Q, K, V}, and f represents the non-linear activation function. The matrix C∈ [symbol] is the learned correlation matrix, which indicates the importance of signals from different reflection areas. Finally, FC refers to the fully-connected layer that projects the weighted value V from N_{b} to 1.

To derive beat-to-beat relations from mm Wave signals reflected by cardiac activities to corresponding ABPWs, this problem is formulated as a time series mapping task. A reliable mapping intends to be consistent across different temporal resolutions.

When the subject's chest is closer to the radar than the diaphragmatic region, it is observed that during chest breathing, a higher concentration of reflection energies is observed in the bins near the radar, whereas during diaphragmatic breathing the energy is more focused on the bins farther away from the radar. Therefore, the spatially-aware attention modules are crucial to concatenate feature maps to avoid information loss at different scales and preserve discriminative features from the same resolution such that effectiveness of the feature extractor across different chest dynamics can be ensured under temporal consistency regularization.

FIG. 8B is an example of feature visualization from the mmFormer feature extractor. The generated feature map has 16 channels, while the lighter color indicates a larger value in the feature map. We can observe that the learned features from the end-to-end neural network are in good agreement with the ground truth in terms of time and amplitude aspects. From the time aspect, the overall pattern in the feature map corresponds well to each cycle of the waveform, indicating good cardiac cycle feature extraction. From the amplitude aspect, the largest value in the feature map occurs in the trough of the ABPW, which indicates the features capture the amplitude derivatives of ABPW.

Personalization is crucial as the individual parameters, such as artery thickness and blood density, vary among different subjects and can significantly impact the model as proved in Equation 3 and 4. Existing PPG-based ABPW estimation personalized waveform regressor suffer from data distribution shifts caused by the inherent diversity of physiological structures, which greatly influences the performance of inter-subject estimation.

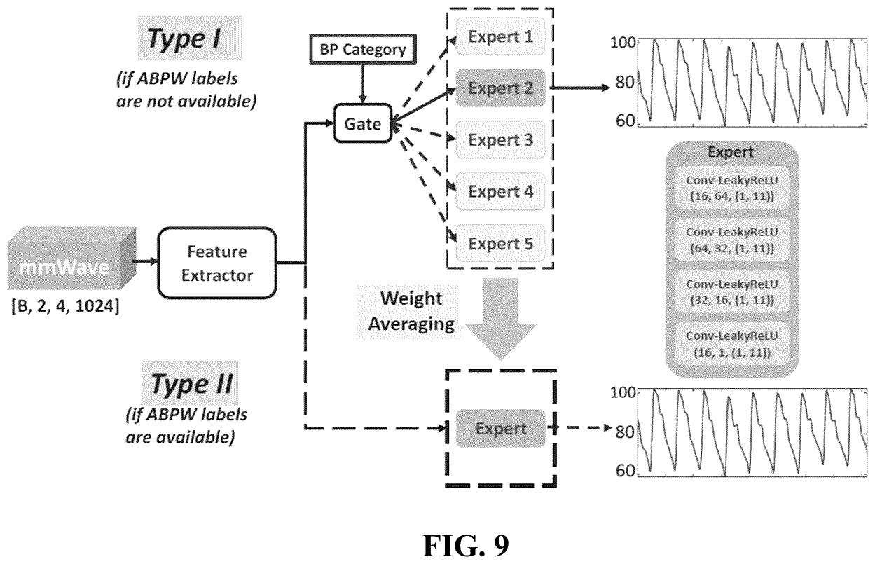

To address the practical issue of data collection burdens and shortcoming of existing personalized waveform regressor, referring to FIG. 9, the mmFormer may further comprise a personalization waveform regressor adopting two model types to address different levels of personalization efforts.

Model Type I is designed for the scenario where the user's ABPW labels are unavailable due to the high cost of ABPW collection devices. In this scheme, we design different expert regressors with an explicit gate on BP categories following the classification by the American Heart Association, i.e., five BP categories, hypotension, normal, elevated, hypertension stage-1, and hypertension stage-2. In the training stage, the feature extractor learns the representations for every input and then forwards them to the corresponding category expert. In the deployment stage, the model only requires knowledge of the coarse BP category, e.g., obtained using a low-cost cuff-based BP monitor, and selects the appropriate regressor for ABPW estimation without tuning the model weights.

Model Type II is designed for scenarios where the user's ABPW data is available. It builds upon the general model trained with different BP categories from Type I. In this case, only one expert regressor is initialized with weight averaging on the five experts learned from the Type I model. After initialization, the Type II model undergoes further training using the user's ABPW data to personalize the model. The personalized model captures individual characteristics to eliminate the need for BP category input during inference.

### BeamDA Module

mmWave signals are susceptible to noises, as demonstrated by the imperfect matching between mmWave phase accelerations and SCG signals in FIG. 7. Based on the observation that vital signals are distributed more consistently than noises across different BeamDA) module beamforming angles, to enhance the model's robustness and mitigate the interference the machine learning tools 180 further include the beamforming-based data augmentation (BeamDA) module trained and configured to steer the complex-valued mmWave IQ data to extract consistent features from different views (angles) to form target signal beams.

The beamforming-based data augmentation module may be configured to employ beamforming techniques to convert raw mmWave signals into a batch of mmWave signal samples with varying beamforming angles to train mmFormer. Signals arriving over different mmWave propagation paths are combined and reception in the desired target direction is reinforced (or focused). Thus, multiple views of the target for data augmentation can be obtained. The augmented data \tilde{X}(θ) for target angles θ are fed into the model separately for training to enforce the consistency regularization across the angles.

Specifically, for a beamforming angle θ, and a minibatch of multi-channel mm Wave data X, the beamforming signal can be represented as follows:

$$\tilde{X} ( θ ) = {W}^{H} ( θ ) X \qquad ( 6 )$$

where W(θ)=[W₁(θ), W₂(θ), . . . , W_{c}(θ), . . . , W_{Nc}(θ)] represents the steering vector of angle θ computed as:

$${W}_{c} ( θ ) = exp [ - j 2 π {d}_{c} sin ( θ ) / λ ] , \qquad ( 7 )$$

where λ is the wavelength of the mmWave and d_{c} is the relative distance introduced by the channel c. Nc is the total number of channels. W^{H}(θ) is the Hermitian transpose (conjugate transpose) of the steering vector.

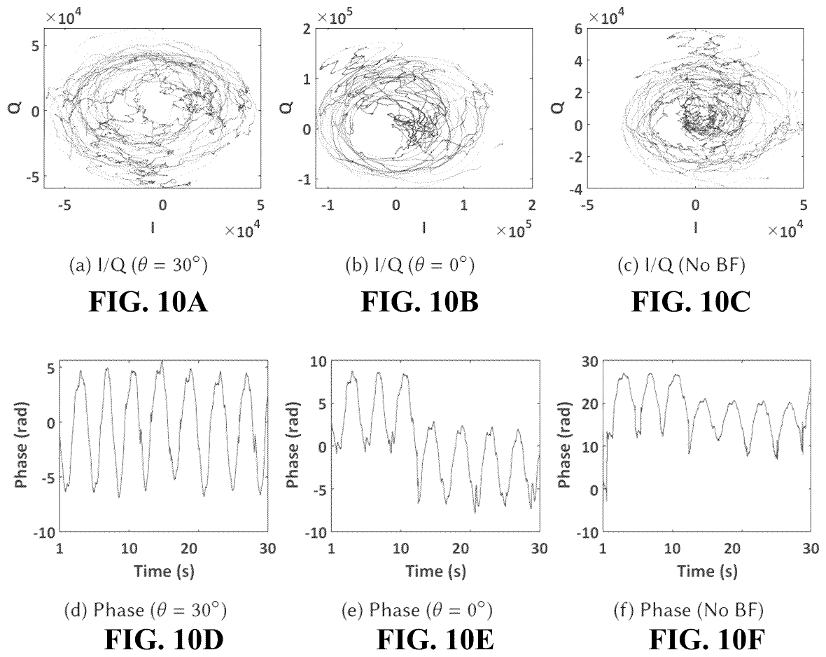

In a case study where a subject sat in front of the radar. The vital signs from two different beamforming angles that maximized the signal strength of the heartbeat frequency bands and the received power are measured, which were set at 30° and 0°, respectively. The beamforming I/Q data results of Barlett beamformer are shown in FIG. 10A to 10C. From FIG. 10D to 10F, the signal phases are influenced by noise to varying extents. However, the overall waveforms induced by breaths (e.g., large cycles) and heartbeats (e.g., peaks) are still preserved. This indicates that noise distributions, such as tremors or unconscious movements of users in different locations, may exhibit differently in different views (angles) of the mm Wave signals. While vital signs, such as breaths and heartbeats, tend to be more consistent. The underlying rationale is that cardiac signals, which are highly correlated with ABPW, tend to exhibit greater consistency across different angles compared to noise signals.

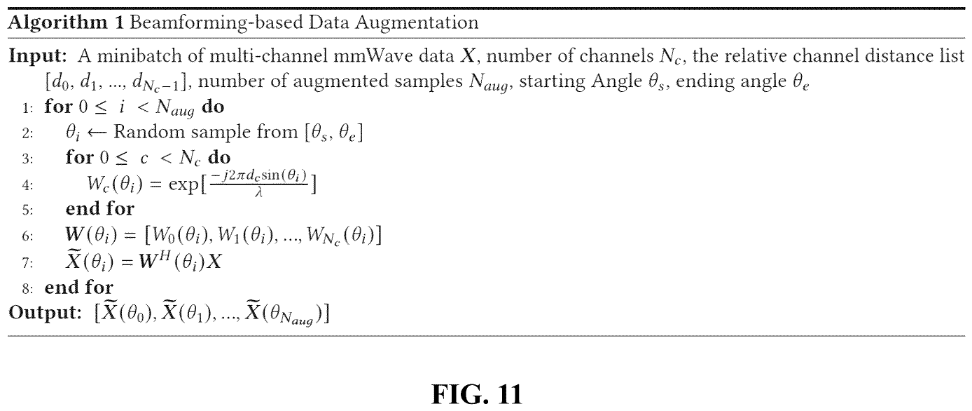

Based on this observation, a BeamDA scheme as outlined in Algorithm 1 as shown in FIG. 11 is devised. In the training stage S102, beamforming using Equation 6 and 7 is applied to the training data with random beamforming angles. Additionally, signals from one antenna without beamforming are included to preserve essential information. I/Q data in FIGS. 10A and 10B can be regarded as augmented examples of the non-beamforming one in FIG. 10C. The training loss is calculated averagely on the augmented inputs on the same ground truth based on the intuition that observations from different views should produce the same results. By training on signals from multiple beamforming angles, the model can effectively retain common cardiac information while mitigating noise and variations effects. In the deployment/testing stage S103, we employ the beamforming algorithm to generate signals that focus on the heart area. Subsequently, we feed them to the mmFormer for further estimation.

The beamforming algorithm exploits the fact that the heartbeat signal is periodic, and it leverages this periodicity in order to identify the best direction of obtaining the corresponding periodic signal. Specifically, the beamforming algorithm decomposes the task of identifying the best beam with the heart rate into two subtasks: a first subtask to solve a 1D CNN-assisted template matching problem; and a second subtask to identify the correct 3D spatial beam and extract its phase.

Accordingly, the beamforming-based data augmentation module is constructed to have an overall architecture consists of two main processing chains: a first chain used to robustly extract the heart rate; and a second chain used to combine beamforming and FFT in order to identify the spectral features coming from each point in 3D space and then use the estimated heart rate from the first chain and the identified spectral features to identify the correct 3D spatial beam.

In one embodiment, the beamforming-based data augmentation module may include a first neural network configured to estimate heart rate of the subject from the complex-valued mmWave IQ data; and a second neural network configured to identify spectral features of the complex-valued mmWave IQ data and identify the target signal beams based on the estimated heart rate and the identified spectral features.

To substantiate the effectiveness of BeamDA, the empirical risk on augmented samples are analyzed with the following equation:

$$\begin{matrix}{ξ}^{BeamDA} ( f ) = {𝔼}_{θ} [ l ( \frac{1}{{N}_{aug}} {\sum }_{i = 0}^{{N}_{aug} - 1} ‖ {\sum }_{j = 0}^{{N}_{c}} {W}^{H} ( {θ}_{j} ) / {N}_{c} ‖ y , \\ \frac{1}{{N}_{aug}} f ( {\sum }_{j = 0}^{{N}_{c}} {W}^{H} ( {θ}_{j} ) {X}_{j} ) ) ] , \\ = {𝔼}_{θ} [ l ( \frac{1}{{N}_{aug}} {\sum }_{j = 0}^{{N}_{c}} ‖ {λ}_{j} ‖ \tilde{y} , \frac{1}{{N}_{aug}} f ( {\sum }_{j = 0}^{{N}_{c}} {λ}_{j} {X}_{j} ) ) ] , \\ where \tilde{y} = \frac{{N}_{aug}}{{N}_{c}} y , {λ}_{j} = {W}^{H} ( {θ}_{j} ) .\end{matrix} \qquad ( 8 )$$

In Equation (8), ξ^{BeamDA}(f) is the empirical risk after BeamDA. f stands for the model to train, i.e. mmFormer. [symbol] is the expectation function on the random-sampled beamforming angles. Xⱼ is the mmWave data from the channel index j while y is the ABPW label. l(⋅, ⋅) is the calculated loss where the first item stands for the label while the second is the input. Note that we use the same ABPW label y for the same mm Wave inputs Xⱼ with different angles and derive the loss with averaging losses from the augmented samples. We could observe that after BeamDA, the empirical risk is a specialized mix-up form that combines inputs from multiple antennas with beamforming weights λⱼ. The empirical risk of Equation (8) has been proved effective in training that help improve robustness and generalization.

### CMKT Module

ABPW also relates to blood volume changes in vessels and detailed cardiac activities, which can be captured by PPG and ECG signals. ECG/PPG signals are highly correlated with ABPW in values and shapes.

Instead of directly feeding ECG/PPG signals into the model, the machine learning tools 180 further include the cross-modality knowledge transfer (CMKT) module configured to facilitate an ECG/PPG-ABPW model acting as a teacher model. The teacher model can supervise mmFormer to extract cardiac-related features that are relevant to ABPW estimation in the training stage S102, that is, to fuse knowledge from ECG-PPG signals with vibrations captured in mm Wave reflections.

The CMKT module facilitates the transfer of learned knowledge from the ECG/PPG-ABPW teacher model to the student model mmFormer. This ensures that mmFormer captures the essential information from the mmWave signals that correlate with cardiac activity. Additionally, mmFormer is also supervised using ABPW labels, which guide the model to regress ABPW values accurately.

In other words, the mmWave model (or student model) is co-supervised by ABPW labels and the ECG/PPG teacher model. It does not require the teacher model and student model to be the same, which brings more flexibility. The teacher model can be trained on a public dataset and fine-tuned on domain-specific dataset, therefore the student model's capability can be implicitly enhanced with a stronger teacher model.

During the deployment stage S103, a beamforming algorithm is applied to the mmWave signals to obtain target signal beams and the target signal beams are fed into mmFormer for ABPW estimation.

The knowledge transfer process only takes place in the training stage without introducing extra overhead in the testing/deployment stage. ECG/PPG signals are only utilized in the training stage, i.e., the requirement for input data is easier and no additional overhead is introduced during deployment. Therefore, the training scheme of mmFormer based on BeamDA and CMKT modules can enhance accuracy without imposing additional efforts on users.

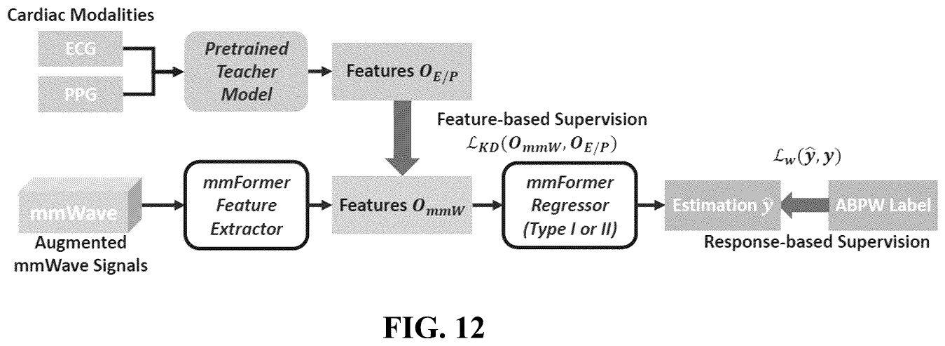

FIG. 12 illustrates schematic diagram of the training scheme for the mmFormer. In the training stage, the ECG-PPG teacher model may be obtained from other sources and aligned with mmWave dataset collected by the mmWave radar in the data collection stage. Its knowledge from intermediate layers is then transferred to the student mmWave model as a regularization to enforce ECG-PPG related feature extraction. In the testing/deployment stage, the mmWave model can work without the ECG-PPG teacher model.

The teacher model's architecture is similar as the student model mmFormer, but the teacher model doesn't require spatially-aware attention as shortcuts to aggregate the encoder's information. ECG/PPG signals are concatenated in the channel dimension and fed to the teacher model. To enhance the teacher model's capability, the teacher model is firstly pre-trained a cleaned version of MIMICII dataset which was collected in the intensive care unit scenarios for 942 patients. Then, the pre-trained teacher model is aligned to the dataset collected by the mm Wave radar by finetuning the first 1×1 convolution kernel, the first layer of the UNet architecture's encoder and decoder, and normalization parameters of the transformer layers since they contain most domain-specific features. All other parameters are frozen.

To minimize the error of estimation results, the following loss function [symbol] is used, which combines the point estimation and the correlation of the whole waveform:

$${ℒ}_{2} ( \hat{y} , y ) = {‖ \hat{y} - y ‖}^{2} \qquad ( 9 )$$

$${ℒ}_{c} ( \hat{y} , y ) = \frac{{\sum }_{i} ( {\hat{y}}^{i} - {μ}_{\hat{y}} ) ( {y}^{i} - {μ}_{y} )}{\sqrt{{\sum }_{i} {( {\hat{y}}^{i} - {μ}_{\hat{y}} )}^{2} {\sum }_{i} ( {( {y}^{i} - {μ}_{y} )}^{2}}} , \qquad ( 10 )$$

$${ℒ}_{w} ( \hat{y} , y ) = α * {ℒ}_{2} ( \hat{y} , y ) + β * {ℒ}_{c} ( \hat{y} , y ) , \qquad ( 11 )$$

where α and β are hyperparameters to balance the loss components, ŷ and y are the estimation and label, μ_{ŷ} and μ_{y} are the mean values of ŷ and y, ŷⁱ and yⁱ are the i-th point in the estimated and label waveforms, respectively.

During training, BeamDA is utilized to generate diverse input samples. To transfer knowledge from the ECG/PPG-ABPW (teacher) model to the mmWave (student) model, for each input sample, mmFormer optimizes towards minimal distance between the estimation and the label as well as minimal distance between the mm Wave model's extracted features and the ECG/PPG-ABPW model's features. The loss function [symbol] for the mmWave model is:

$${ℒ}_{D} ( {O}_{mmW} , {O}_{E / P} ) = {‖ {O}_{mmW} - {O}_{E / P} ‖}^{2} , \qquad ( 12 )$$

$$ℒ ( \hat{y} , y , {O}_{mmW} , {O}_{E / P} ) = α * {ℒ}_{2} ( \hat{y} , y ) + β * {ℒ}_{c} ( \hat{y} , y ) + γ * {ℒ}_{KD} ( {O}_{mmW} , {O}_{E / P} ) , \qquad ( 13 )$$

where O_{mmW} and O_{E/P} are the extracted features from the mmWave model and teacher model as shown in FIG. 12. In this setting, the extracted features from the output of the mmFormer feature extractor, i.e., the first layer of the UNet-based mmWave model and ECG/PPG-ABPW model decoders are selected as O_{mmW} and O_{E/P}, respectively.

### Implementation

The mmWave radar of the contactless ABPW monitoring system 100 may be implemented with a COTS mmWave radar and evaluated with a TI IWR1443BOOST board and a TI DCA1000EVM board). The radar operates on the 77 GHz with a bandwidth of 4 GHz. This radar contains 3 transmitting antennas and 4 receiving antennas. The radar transmits signals with two transmitting antennas simultaneously for higher transmission power and receive signals by four receiving antennas. The mmWave device sends one frame for every T_{f}=0.002 s. We use one chirp in each frame with 256 sampling points.

The machine learning tools of the system 100 may be implemented with one NVIDIA RTX 3090 GPU based on PyTorch 1.11.0. The ECG/PPG teacher model is firstly pre-trained on a cleaned MIMICII dataset containing ECG, PPG and ABPW with the preprocessing techniques and then transferred to the collected dataset with a batch size of 128 and learning rate of 1e⁻⁴ in 10 epochs. We downsample the sampling rate from 500 Hz to 125 Hz to align with the sampling frequency of MIMICII. The size of the mm Wave data X∈ [symbol] before BeamDA is set as N_{c}=4, N_{b}=4, and N_{f}=1024. Sliding window of 128 is used along the slow time axis to create the training samples. The ECG/PPG teacher model, the mmWave Type I model, and the personalized Type II model have initial learning rates of 1e⁻³, 2e⁻⁴ and 5e⁻⁵, respectively. All learning rates decay in a cosine manner to its 1/20 in 25 epochs. We train the models with the Adam optimizer and a batch size of 64. For training the BeamDA module, we augment the mm Wave signals by randomly selecting 4 angles within −60° and 60° with a step of 10° considering the radar's angular resolution.

Referring back to FIGS. 8A and 8B, in the mmFormer's feature extractor, the number of input channels, output channels, and kernel size of the first IQ-convolution kernel to fuse I/Q data on the first layer are 2, 16, and (1, 1) respectively. All other convolution kernels have a kernel size of (1, 11). The numbers of output channels of UNet-convolution kernels, Downsample-convolution kernels, and spatially-aware attention modules of mmFormer's encoder are 16, 32, 64, and 128, respectively. The last layer contains a convolution kernel with 256 output channels, followed by transformer layers. The number of the transformer layer is 12 and each layer has 8 heads. The numbers of output channels of UNet-convolution kernels and Upsample-convolution kernels of mmFormer's decoder are 128, 64, 32, and 16, respectively. The numbers of output channels of each regressor's convolution kernels are 64, 32, 16, 1. All the normalization modules are InstanceNorm. All the nonlinear activation functions are LeakyReLU with a negative slope of 0.3. For the loss function, we empirically set the loss weight parameters as α=1, β=100, γ=50.

### Evaluation

The contactless ABPW monitoring system 100 provided by the present invention is evaluated on a dataset of 43 subjects (20 females and 23 males) including 4 with hypotension, 24 with normal, 3 with elevated, 10 with hypertension stage-1, and 2 with hypertension stage-2 ABPW readings, respectively. All participants self-reported no cardiac disease.

Type I and Type II schemes are evaluated separately to fully assess the performance of the contactless ABPW monitoring system.

For the Type I scheme, we use a leave-one-subject-out (LOSO) setup to evaluate the contactless ABPW monitoring system, which involves splitting the 43-subject dataset into two parts: one contains 42 subjects for training and the other contains one subject for testing. In the Type I scheme, the model is only aware of the test subject's coarse BP category for testing rather than the detailed ABPW data. This setup assesses the model's ability to generalize to unseen subjects and is more applicable to real-world scenarios.

For the Type II scheme, we personalize the basic model from Type I with the target user's data. To avoid samples with adjacent timesteps split into train/test sets, we retrain the model acquired from the Type I scheme with the random 70% of collected trials and then test its performance for the other 30% trials. The average number of samples for each subject is 866 (train) and 435 (test).

To measure performance of the contactless ABPW monitoring system, three levels of evaluation metrics as shown in Table 3 are employed. The first level assesses point estimation, including mean error (MEp), standard deviation (STDp), and mean absolute error (MAEp) The second level uses the Pearson Correlation Coefficient (PCC_{w}) to compare the estimated waveforms with the references. For the third level, we choose the commonly used mean error (MEₛ), the standard deviation of the error (STDₛ), and Pearson Correlation Coefficient (PCCₛ) of the discrete BP values, i.e., SBP and DBP. Where L is the length of the waveform, Nis the total number of waveforms, M=N×L is the total number of blood pressure points, and the mean values of ŷ, y, \hat{ξ}, ξ are μ_{ŷ}, μ_{y}, μ_{\hat{ξ}}, μ_{ξ}, respectively.

TABLE 3
Evaluation metrics.

| Level | Point | Statistic | Waveform |
|---|---|---|---|
| | ${ME}_{p} = \frac{{\sum }_{i = 1}^{M} ( {\hat{p}}_{i} - {p}_{i} )}{M}$ | ${ME}_{s} = \frac{{\sum }_{k = 1}^{N} ( {\hat{υ}}_{k} - {υ}_{k} )}{N}$ | |
| Metric Formula | ${STD}_{p} = \sqrt{\frac{{\sum }_{i = 1}^{M} {( {\hat{p}}_{i} - {p}_{i} - {ME}_{p} )}^{2}}{M}}$ | ${STD}_{s} = \sqrt{\frac{{\sum }_{k = 1}^{N} {( {\hat{υ}}_{k} - {υ}_{k} - {ME}_{s} )}^{2}}{M}}$ | ${PCC}_{w} = \frac{{\sum }_{j = 1}^{L} ( {\hat{y}}^{j} - {μ}_{\hat{y}} ) ( {y}^{j} - {μ}_{y} )}{\sqrt{{\sum }_{j = 1}^{L} {( {\hat{y}}^{j} - {μ}_{\hat{y}} )}^{2} {\sum }_{j = 1}^{L} ( {( {y}^{j} - {μ}_{y} )}^{2}}}$ |
| | ${MAE}_{p} = \frac{{\sum }_{i - 1}^{M} [ ( {\hat{p}}_{j} - {p}_{i} ) ]}{M}$ | ${PCC}_{s} = \frac{{\sum }_{k = 1}^{N} ( {\hat{υ}}_{k} - {μ}_{\hat{o}} ) ( {υ}_{k} - {μ}_{o} )}{\sqrt{{\sum }_{k = 1}^{M} {( {\hat{υ}}_{k} - {μ}_{\hat{o}} )}^{2} {\sum }_{k = 1}^{N} ( {( {υ}_{k} - {μ}_{o} )}^{2}}}$ | |

The results under the leave-one-subject-out (LOSO) setting validate that the present invention can achieve a high waveform correlation of 0.903 and a low-value error (mean±standard deviation) of each point of (−0.14±7.48) mmHg. SBP and DBP measurements from reconstructed ABPW have low errors of (−1.80±7.02) mmHg and (0.12±5.40) mmHg, respectively, within the Association for the Advancement of Medical Instruments (AAMI) requirement (5±8) mmHg. Real-world applicability was validated through three case studies showing that the present invention holds great potential for fine-grained cardiac indicator estimation, cardiac abnormality detection, and continuous long-time monitoring.

### Case Studies

To validate significance and applicability, we conduct three case studies. Case Studies 1 and 2 show the significance of ABPW by its potential for fine-grained cardiac indicator estimation and cardiac abnormality detection. Case study 3 presents the applicability of a usage scenario that monitors a subject's ABPW while watching TV without any disturbances.

### Case Study 1: Relative Cardiac Output Through ABPW

To illustrate the importance of ABPW, we conduct a case study to calculate the relative cardiac output using the estimated ABPW from the two schemes and the reference ABPW. The relative cardiac output error is defined as

$$r = \frac{\vert G ( \hat{y} ) - G ( y ) \vert}{G ( y )} ,$$

where G is the algorithm to G (y) estimate CO from ABPW, ŷ and y are the estimated ABPW and the reference ABPW, respectively.

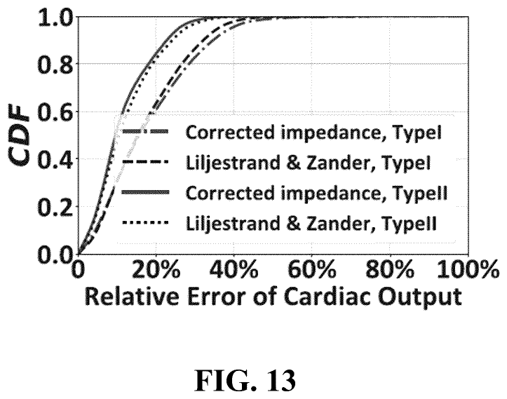

We calculate the relative error r from the estimated results of the 43 subjects based on the two best CO estimators from the previous work, including Liljestrand & Zadnder formula and corrected impedance formula, as shown in FIG. 13. The mean errors of the relative cardiac output results for Type I and Type II are 17.1% and 18.0% with the Liljestrand & Zadnder formula and 11.4% and 12.3% with the corrected impedance formula, respectively. This low error indicates that the CO from ABPW estimated by the present invention has a good agreement with that from the reference ABPW. Despite the intrinsic error from the CO estimator, it shows a promising direction that we could further leverage the ABPW sensed by the present invention to monitor fine-grained hemodynamic indicators for preventing heart diseases in a contactless manner.

### Case Study 2: Cardiac Abnormality Detection Through ABPW

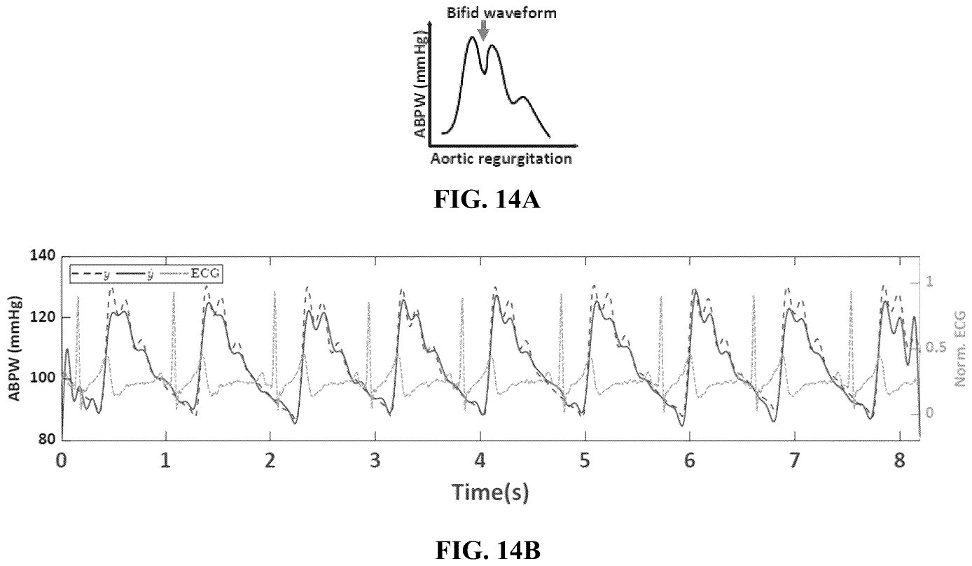

Compared with discrete BP values, ABPW reflects cardiac abnormalities. To validate its effectiveness, we collect data from a subject with chronic aortic regurgitation (AR), which is a type of heart valve disease. AR indicates that the valve between the lower left heart chamber and the main artery of the body does not close tightly, causing the blood pumped out of the left ventricle to leak backward. This disease may lead to pulmonary edema and even cardiogenic shock. Compared with ECG which may be non-specific to AR, ABPW is a good indicator of AR with widened pulse pressure. During the cardiac cycle, the blood will first be pumped out of the left ventricle with a high peak in ABPW. Then the blood that leaks backward due to AR causes the second peak, resulting in the bifid waveform pattern as shown in FIG. 14A. The discrete SBP/DBP of this subject is 131/86 mmHg, only indicating the hypertension stage-1, while his ABPW indicates finer-grained cardiac dynamics with abnormal blood leaking backward as shown in FIG. 14A. Compared with ground truth, the ABPW estimated by the present invention (with Type II personalization) in FIG. 14B can well capture the bifid waveform feature with a high PCCw of 0.971 and a low MAEp of 2.28 mmHg, confirming the potential for the detection of cardiac abnormalities through contactless ABPW monitoring.

### Case Study 3: Long-Time Monitoring for a User Watching TV

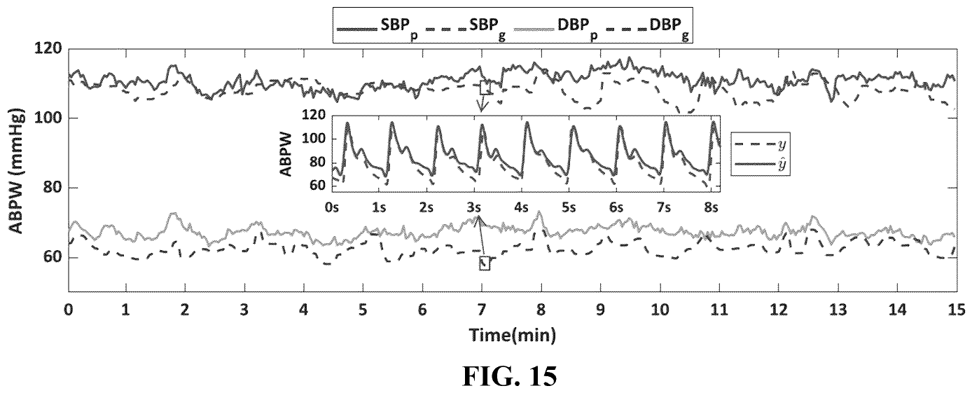

To validate the real-world applicability, one volunteer is asked to watch TV in front of the radar which is placed 50 cm in front of him. We use 18 minutes of data for Type II personalization and then deploy the radar to track his ABPW in a contactless manner. The only requirement is to avoid large motions which is for both our system and the reference equipment. As shown in FIG. 15, ABPW estimations by the present invention are consistent with the ground truth, which indicates that the present invention can accurately track a user's BP variation. Specifically, The MAE of SBP and DBP predictions are 3.44 mmHg and 4.81 mmHg, respectively. Besides a low error of discrete SBP and DBP estimations, the present invention can achieve a low MAE error of point-level predictions as 4.80 mmHg and a high waveform correlation of 0.958. This case study mimics a typical usage scenario for the present invention and validates its applicability for long-time non-intrusive monitoring.

The functional units and modules in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

While the present disclosure has been described and illustrated with reference to specific embodiments thereof, these descriptions and illustrations are not limiting. The illustrations may not necessarily be drawn to scale. There may be distinctions between the illustrations in the present disclosure and the actual apparatus due to manufacturing processes and tolerances. There may be other embodiments of the present disclosure which are not specifically illustrated. Modifications may be made to adapt a particular situation, material, composition of matter, method, or process to the objective and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. A machine learning-based system for contactless ABPW monitoring, comprising:
a mmWave radar;
one or more processors in communication with the mmWave radar;
machine learning tools in communication with the one or more processors;
a memory device storing the machine learning tools and inter-operably coupled with the one or more processors;
wherein:
the machine learning tools include an mmWave-ABPW transformer and a beamforming-based data augmentation module;
the mm Wave radar is configured to transmit a mm Wave signal waveform and to receive reflection signals from a subject's chest;
the memory device further stores instructions that, when executed, cause the one or more processors to:
extract complex-valued mm Wave IQ data from the received reflection signals;
utilize the beamforming-based data augmentation module to steer the complex-valued mmWave IQ data to form target signal beams; and
utilize the mmWave-ABPW transformer to estimate ABPW from the target signal beams.

2. The machine learning-based system of claim 1, wherein:
the transmitted mmWave signal waveform is a frequency modulated continuous waveform; and
the mm Wave radar is further configured to mix the received reflection signals with the transmitted mmWave signal waveform to obtain intermediate frequency signals.

3. The machine learning-based system of claim 2, wherein the one or more processors are configured to:
apply range-fast Fourier transformation on the intermediate frequency signals to obtain frequency and phase of the intermediate frequency signals;
detect the signal regions of the subject's chest from the frequency and phase of the intermediate frequency signals using a constant false alarm rate algorithm;
search range bins with largest reflection energy from the detected signal regions; and
extract the complex-valued mm Wave IQ data from the searched range bins.

4. The machine learning-based system of claim 1, wherein the mmWave-ABPW transformer includes a ABPW feature extractor comprising:
an encoder configured to encode the target signal beams and generate a compressed feature tensor;
a self-attention transformer configured to perform global context modelling and spatial attention refinement on the compressed feature tensor to obtain a refined feature tensor;
a decoder configured to decode the refined feature tensor and generate an ABPW feature tensor; and
a plurality of spatially-aware attention modules bridging intermediate layers of the encoder and the decoder to facilitate the ABPW feature extractor to concatenate feature tensors at different spatial dimensions by selectively re-weight feature activations according to importances of the target signal beams from different reflection areas.

5. The machine learning-based system of claim 4, wherein the mmWave-ABPW transformer further includes a personalization waveform regressor comprising:
a plurality of expert regressor models trained respectively with a plurality of training data sets corresponding to respective blood pressure categories; and
an explicit gate configured to select an appropriate expert regressor model for ABPW estimation based on a coarse blood pressure category of the subject.

6. The machine learning-based system of claim 5, wherein the appropriate expert regressor model is further trained using ABPW data of the subject.

7. The machine learning-based system of claim 1, wherein the beamforming-based data augmentation module comprises:
a first neural network configured to estimate heart rate of the subject from the complex-valued mmWave IQ data; and
a second neural network configured to identify spectral features of the complex-valued mmWave IQ data and identify the target signal beams based on the estimated heart rate and the identified spectral features.

8. The machine learning-based system of claim 1, wherein the beamforming-based data augmentation module is configured to generate mmWave signal beam data from different angles for training the mmWave-ABPW transformer.

9. The machine learning-based system of claim 1, wherein the machine learning tools further include a cross-modality knowledge transfer module configured to generate a teacher model to co-supervise training of the mmWave-ABPW transformer.

10. The machine learning-based system of claim 1, wherein the teacher model is a transformer model trained on a public ECG/PPG dataset to predict ECG/PPG signals from mmWave data.

11. A machine learning-based method for contactless ABPW monitoring, comprising:
utilizing a mmWave radar to transmit a mmWave signal waveform and to receive reflection signals from a subject's chest;
extracting complex-valued mmWave IQ data from the received reflection signals;
utilizing a beamforming-based data augmentation module to steer the complex-valued mmWave IQ data to form target signal beams; and
utilizing a mmWave-ABPW transformer to estimate ABPW from the target signal beams;
wherein the beamforming-based data augmentation module and the mmWave-ABPW transformer are machine learning tools in communication with one or more processors and stored in a memory device inter-operably coupled with the one or more processors.

12. The machine learning-based method of claim 11, wherein:
the transmitted mmWave signal waveform is a frequency modulated continuous waveform; and
the machine learning-based method further comprises mixing the received reflection signals with the transmitted mmWave signal waveform to obtain intermediate frequency signals.

13. The machine learning-based method of claim 12, wherein the complex-valued mmWave IQ data is extracted by the one or more processor through:
applying range-fast Fourier transformation on the intermediate frequency signals to obtain frequency and phase of the intermediate frequency signals;
detecting the signal regions of the subject's chest from the frequency and phase of the intermediate frequency signals using a constant false alarm rate algorithm;
searching range bins with largest reflection energy from the detected signal regions; and
extracting the complex-valued mmWave IQ data from the searched range bins.

14. The machine learning-based method of claim 11, wherein
the mmWave-ABPW transformer includes a ABPW feature extractor comprising: an encoder, a self-attention transformer, a decoder and a plurality of spatially-aware attention modules bridging intermediate layers of the encoder and the decoder;
the estimation of the ABPW from the target signal beams comprises:
utilizing the encoder to encode the target signal beams and generate a compressed feature tensor;
utilizing the self-attention transformer to perform global context modelling and spatial attention refinement on the compressed feature tensor to obtain a refined feature tensor;
utilizing the decoder to decode the refined feature tensor and generate an ABPW feature tensor; and
utilizing the plurality of spatially-aware attention modules to facilitate the ABPW feature extractor to concatenate feature tensors at different spatial dimensions by selectively re-weight feature activations according to importances of the target signal beams from different reflection areas.

15. The machine learning-based method of claim 14, wherein
the mmWave-ABPW transformer further includes a personalization waveform regressor comprising a plurality of expert regressor models;
the machine learning-based method further comprises
training the plurality of expert regressor models respectively with a plurality of training data sets corresponding to respective blood pressure categories; and
selecting, among the plurality of trained expert regressor model, an appropriate expert regressor model for ABPW estimation based on a coarse blood pressure category of the subject.

16. The machine learning-based method of claim 15, further comprising training the appropriate expert regressor model using ABPW data of the subject.

17. The machine learning-based method of claim 11, wherein
the beamforming-based data augmentation module comprises a first neural network and a second neural network; and
the target signal beams are formed by:
utilizing the first neural network to estimate heart rate of the subject from the complex-valued mmWave IQ data; and
utilizing the second neural network to identify spectral features of the complex-valued mmWave IQ data and identify the target signal beams based on the estimated heart rate and the identified spectral features.

18. The machine learning-based method of claim 11, further comprising utilizing the beamforming-based data augmentation module to generate mmWave signal beam data from different angles for training the mmWave-ABPW transformer.

19. The machine learning-based method of claim 11, further comprising utilizing a cross-modality knowledge transfer module to generate a teacher model to co-supervise training of the mmWave-ABPW transformer.

20. The machine learning-based method of claim 11, wherein the teacher model is a transformer model trained on a public ECG/PPG dataset to predict ECG/PPG signals from mmWave data.
